(19) United States
(12) Patent Application Publication
(10) Pub. No.: US 2023/0165794 A1
(43) Pub. Date: Jun. 1, 2023

(54) OMEGA FATTY ACIDS-RICH OIL DISPERSION FORMULATIONS

(71) Applicant: BIOPLUS LIFE SCIENCES PVT. LTD., Mumbai, Maharashtra (IN)

(72) Inventors: Sundeep AURORA, Bangalore, Karnataka (IN); Neha AURORA, Bangalore, Karnataka (IN)

(21) Appl. No.: 17/922,771

(22) PCT Filed: May 3, 2021
(86) PCT No.: PCT/IN2021/050429

§ 371 Date: Nov. 1, 2022

(30) Foreign Application Priority Data
May 2, 2020 (IN) 201921044492

(51) Int. Cl. A61K 9/107; A61K 31/202; A61K 9/00; A61K 36/02; A61K 47/36

(52) U.S. Cl. CPC A61K 9/1075; A61K 31/202; A61K 9/0053; A61K 36/02; A61K 47/36

(57) ABSTRACT

The invention comprises an oral Omega-3-fatty acid-rich oil emulsion composition comprising DHA 50-100 mg/ml, made from all natural and biocompatible ingredients for use in management of treatment of a disorder to overcome the same. The composition may be a thixotropic emulsion in nanometric size range having better absorption, better stability at room temperature and refrigerated one (2-8° C.), either alone or in combination with vitamins, minerals, Generally Regarded As safe (GRAS) natural ingredients. in therapeutically effective amount. The disorders comprise pre-term birth disorder in pregnant women, cognitive disorders in children and cardiovascular disorders. The said emulsion is made by adding an oil phase comprising a natural emulsifier to an aqueous phase comprising a gum. Vitamin E TPGS, a preservative and a high intensity sweetener; under mechanical stirring. This invention also embodies a High Performance Liquid Chromatography (HPLC) method for assay of omega-3 fatty acids.

### FIELD OF INVENTION

The present invention relates to a stable and effective dispersion formulations of Omega 3 fatty acid (O3FA)-rich oil for oral administration in infants, children's, adult and food fortifications.

### BACKGROUND OF INVENTION

Docosahexanoic acid (DHA) is an essential omega fatty acid found in neuronal and other body tissues. Proper development of neurological tissues and cognitive skills is highly dependent on adequate intake of omega-3 fatty acids especially DHA in the diet. As the brain mass increases approximately 3.5 times upto the age of 5 years, it requires the accumulation of Omega-3 fatty acid (O3FA). Thus the intake of O3FA (500 mg/day) from different dietary source is highly recommended during pregnancy and infancy even in adulthood by American Dietetic Association (Kris-Etherton, P M. and Innis, S. Position of the American Dietetic Association and Dietitian of Canada: Dietary fatty acids, J. Am. Diet Assoc., 2007, 107(9), 1599-611). Large number of children are affected worldwide with cognitive disorder and poor neurological conditions, Attention Deficit Hyperactivity Disorder (ADHD) is one of the most common cognitive disorders of childhood. In the United States approximately 8-10% children are affected with this disorder and in India the prevalence is even higher to 5-29% (http://www.slideshare.net/adhdarabia/adhd-facts-and-figures). Also an increasing trend in loss of learning disability is an alarming concern towards the cognitive health of children; about 2.4 million children are affected with this disorder in US (National center of learning disabilities, 2014). Omega fatty acid (O3FA) is highly lipophilic in nature. For the purpose of this specification “Omega Fatty Acid” or “O3FA” is defined as a fatty acid which has at least one double bond in its carbon backbone. O3FA includes, without limitation, Omega-3 fatty acid and Omega-6 fatty acid and Omega-9 fatty acid or a combination thereof. Examples of O3FA includes Docosahexaenoic acid (DHA), Eicosapantaenoic acid (EPA) Docosapantaenoic acid (DPA) and/or Arachidonic acid; or combination thereof. Converting O3FA in order to enhance its applicability into water soluble products is a challenging task. Presently, available formulation like soft gelatin capsules are the most commonly used dosage form of O3FA-rich oils, which suffer from their own disadvantages; like obnoxious refluxes, flatulence, low bioavailability and the unsuitability of administration to infant and children. Moreover, the oral liquid formulations of O3FA-rich oils are limited and even these formulations further suffers from poor dispersibility and absorption in GIT fluid, short storage shelf life, oxidative instability and poor organoleptic profile. Thus poor patient compliance and the lack of stable and therapeutically effective formulation in children remain primary hurdles for widespread clinical use of O3FA-rich oil.

In this invention a pharmaceutically stable and organoleptically elegant O3FA-rich oil emulsion formulation with increased bioavailability, oxidative stability, enhanced storage life and enhanced therapeutic index has been developed. Further the safety profile is proven by cell line studies.

The present invention describes an oral thixotropic emulsion in nanometric size range of O3FA-rich oil. The said thixotropic emulsion composition of O3FA-rich microbial oil is stable and therapeutically efficacious. This invention also describes process of preparation of the thixotropic emulsion composition of O3FA-rich microbial oil. The composition is optionally fortified with vitamins and minerals.

O3FA in general and O3FA especially are the biomolecules implicated for proper development of the brain and other neurological conditions. O3FA deficiency is also associated with several medical disorders including enhanced risk of pre-term birth. But due to its highly lipophilic oily nature, O3FA suffers from poor water dispersibility, poor organoleptic profile and oxidative instability. Presently, only limited oral formulations of O3FA-rich oils are available under the category of nutraceuticals. They possess limitations of short shelf life and poor oxidative stability along with separation of oil during storage. In this invention, a stable thixotropic emulsion formulation is developed for oral administration of O3FA. The stable emulsion formulation is optionally fortified with vitamins and minerals. Therapeutics effectiveness and toxicity profile of the thixotropic emulsion formulation of this invention has been demonstrated in vivo and Ex vivo.

Emulsion is a biphasic colloidal system comprising of oil phase, water phase, emulsifiers and stabilizers. O/W emulsion (Oil-in-water) emulsion is a best suited formulation for oral administration of lipophilic active moieties i.e. drugs, oils, Vitamins etc., in which the oil phase is well dispersed as oil globules in continuous water phase, stabilized by different emulsifiers and stabilizers. It was surprisingly found that whereas emulsions of O3FA-rich oil are physically unstable wherein under storage the oil separates after some time and it is also oxidatively unstable, upon transforming the same in a thixotropic system (gel-sol-gel), the resulting composition led to physical stability as well as oxidative stability, resulting in increased storage shelf life. Further, this delivery system has flexibility to include different sweetening and flavoring agents which increases the palatability during oral administration. Further this is easy to scale up as the preparation described therein is simple and easy. Thus, this invention has provided a method and a system wherein palatable and stable compositions can be done from O3FA rich oil for nutritional therapy for health benefits brought in by O3FA.

DHA is a one of the most widely used O3FA plays an important role for the effective management of pre-term birth disorder. In this invention application of developed dispersive formulation for the treatment of pre-term birth disorder has been disclosed.

Preterm birth (<37 weeks of gestation) is one of the leading causes of infants death worldwide. It accounts about 17% of deaths in children under 5 years of age and more than 85% of all prenatal complications (Makrides M, Best K. Docosahexaenoic acid and preterm birth. Annals of Nutrition and Metabolism. 2016; 69(Suppl. 1):29-34.). Advancement in perinatal and neonatal care will decrease the number of preterm births cases and improve cognition disorders in infants. Epidemiological and randomized trial studies have observed an increased length of gestation, infant weight and head circumference at birth in populations with high fish consumption (Greenberg J A, Bell S J, Van Ausdal W. Omega-3 fatty acid supplementation during pregnancy. Reviews in obstetrics and Gynecology. 2008; 1(4):162.; 3. Baack M L, Puumala S E, Messier S E, Pritchett D K, Harris W S. What is the relationship between gestational age and docosahexaenoic acid (DHA) and arachidonic acid (ARA) levels?. Prostaglandins, Leukotrienes and Essential Fatty Acids. 2015 Sep. 1; 100:5-11.). Olson and Joensen first observed that Faroe Islanders, who consume more long chain polyunsaturated fatty acids (LCPUFA) such as docosahexaenoic acid (DHA) and eicosapentaenoic acid (EPA) rich seafood than do Danes, had a longer gestation duration and infants with higher birth weights.

Despite enormous information about the pathophysiology of pregnant women and preterm births, currently there is no strategy that can be used as a primary prevention for widespread clinical use. The prenatal period is highly vulnerable window that is sensitive to DHA deficiency. Omega-3 fatty acids are essential and can only be obtained from the diet. These LCPUFA including DHA are increasingly transferred from mother to foetus late in pregnancy (3rd trimester). Infants born before this transfer is at risk for deficiency. The requirements during pregnancy have not been established, but likely exceed that of a non-pregnant state. These fatty acids are critical for foetal neurodevelopment especially visual and neural function (Rogers L K, Valentine C J, Keim S A. DHA supplementation: current implications in pregnancy and childhood. Pharmacological research. 2013 Apr. 1; 70(1):13-9.). According to the National Institute of Health, the consumption of DHA is poor in developed as well as low developed countries. Average intake is 40 mg in children and teens and about 90 mg in adults. Recommendations for DHA are currently set at a minimum of 200-300 mg/day for pregnancy and lactation. However, some studies have shown that supplementation with less than 600 mg/day were not beneficial in preventing early preterm birth. DHA supplementation in the range of 600 mg-1000 mg has been associated with decreased risk of pre-term delivery and higher birth weight, as well as a positive effect on infant's brain development (https://clinicaltrials.gov/ct2/show/NCT02626299?term=DHA+800+mg&cond=Preterm+birt&draw=2&rank=1).

Most pregnant women do not get enough omega-3 fatty acids because the major dietary source, seafood, is restricted to two servings a week. For pregnant women to obtain adequate omega-3 fatty acids, a variety of sources should be consumed viz vegetable oils, low-mercury fish servings a week, and supplements (fish oil or algae-based docosahexaenoic acid). Given this emerging data, pregnant women of all dietary patterns will likely benefit from consuming a daily DHA supplement from either fish oil or algae oil between 600-1000 mg/day. Algal oil possess a advantage over fish oil as it is derived from microalgae, which is what fish consume to get their DHA, and is biologically equivalent to the DHA available in fish oil.

Cognitive disorders are a category of mental health disorders that primarily adversely affect learning, memory, perception, and problem solving abilities in children. These disorders range from deep intellectual impairments to mild impairment in specific activities. These disorders generate due to low or improper intake of O3FA during brain development phases of life. Most commonly used dosage form as a source of O3FA is soft gelatin capsules of O3FA-rich oils. but they are not suitable for administration to infants and children's and even for adults due to obnoxious refluxes and low bioavailability. Further limited availability and poor shelf life with low O3FA content of liquid formulation urges a need to develop stable and therapeutically effective formulation containing O3FA moieties.

### PRIOR ART

US Patent 2006/0165735A1 A1 discloses an oil emulsion, comprising: an oil component comprising polyunsaturated fatty acids; an emulsifier; an emulsion stabilizer; and water; wherein the oil emulsion has not been heat treated. The physical stability at room temperature and 4 C was claimed for only 180 days. No claim for further storage was made.

US patent 2012/0251685A1 A1 discloses an oil-in-water emulsion comprising: a) an oil containing a polyunsaturated fatty acid; b) an emulsifier; c) water; d) a metal chelating agent; and e) an antioxidant; wherein the metal chelating agent is present in an amount from about 3% to about 20% by weight of the emulsion and wherein the antioxidant is present in an amount from about 2% to about 20% by weight of the emulsion. Storage shelf life was claimed upto five to six months at refrigerated conditions. The use of high concentration of metal chelating agent and antioxidants upto 20% is considerably more than prescribed limits.

US patent 2011/0054029A1 A1 discloses a water-soluble dietary fatty acid gel formulation, comprising: from 1 wt % to 75 wt % of dietary fatty acid; and from 25 wt % to 99 wt % of non-ionic surfactant. Use of non-ionic surfactant hydrogenated castor oil/macrogolglycerol hydroxystearate (Cremophor RH 40) may have health related side effects like vasodilation, nephrotoxicity etc when consumed at high concentration.

US 20120093998A1 discloses an emulsion comprising (i) 5-20 weight-% (wt-%), based on the total weight of the emulsion, of PUFA, and (ii) 10-40 wt-%, based on the total weight of the emulsion, of at least one emulsifier, which is a polymeric hydrocolloid originated from a plant source, (iii) 5-45 wt-%, based on the total weight of the emulsion, of at least one adjuvant, and (iv) 15-50 wt-%, based on the total weight of the emulsion, of water. However, no details of globule size, physical and chemical stability of prepared formulation are given

U.S. Pat. No. 9,302,017B2 discloses the micellar formulation of omega 3 fatty acid ester that from average diameter of from about 1 μm to 10 μm. The micellar formulation was totally based on synthetic surfactants Polysorbate 80 and Pluronic F87 using Fish oil. There was no detailed physical and chemical stability of prepared formulation performed and strength in terms of omega 3 fatty acids was also not disclosed. Inventor claimed the application of this formulation in maintenance of cardiovascular health.

US 2011/0200644A1 discloses an emulsion comprising an emulsifier, an isotonic agent and an oil comprising docosahexaenoic acid ethyl ester (DHA-EE), wherein the emulsion is substantially free of eicosapentaenoic acid (EPA) and is suitable for parenteral administration, The O3FA ester emulsion formation was based on Gelatin and Lipoid E80 SN. Detailed physical and chemical stability of prepared formulation was not performed. Inventor claimed the application of this formulation in maintenance of inflammatory conditions.

US patent 2012/0308704 A1 discloses an emulsion as an ingredient or additive for producing food products including omega-3 fatty acids, the emulsion comprising: an outer water phase including at least one water soluble antioxidant dissolved in water; and an inner fat or oil phase which includes plant oil droplets provided with at least one fat or oil soluble antioxidant and with an omega-3 fatty acid ester, wherein the plant oil droplets are provided with a shell made from plant protein. The emulsion was based using Pea protein isolate as emulsifier and fish oil as a source of omega 3 fatty acids. The developed formulation was claimed mainly for food fortifications especially for production of poultry beef sausage with 1% content of omega 3 fatty acid.

U.S. Pat. No. 9,532,963B2 disclosed a food supplement or nutritional supplement composition comprising: a fatty acid oil mixture comprising from about 25% to about 75% eicosapentaenoic acid (EPA) and docosahexaenoic acid (DHA), by weight of the fatty acid oil mixture, wherein the EPA and DHA are in a form chosen from methyl ester, ethyl ester, and triglyceride; and at least one free fatty acid chosen from EPA, DHA, ALA, HPA, DPA, ETA, ETE, STA, linoleic acid, GLA, AA, osbond acid, oleic acid, ricinoleic acid, erucic acid, and mixtures thereof. Formulations were totally based on synthetic surfactants such as Cremophor, Pluronic, Briz. Inventor claimed the application of this formulation in maintenance of cardiovascular health.

Thus, in none of the prior arts, problem of pre-term birth was investigated nor solved.

None of the emulsions carried a strength of DHA in more than 25 mg/ml that is required for therapeutic efficacy for children in a couple of ml. Unless therapeutically effective dose could be carried in reasonably small quantity of emulsion the same is not useful as practical dosage form. Further, prior art compositions have synthetic surfactants, which are contraindicated for administration in Children including neonates. Further, prior art emulsion dispersed phase is in micrometer range, which has limited absorption of DHA. Prior art emulsions have poor stability at room temperature. Further, preparation methods are very tedious and costly.

Hence, there was a need of improved emulsions with better and improved method of their preparation.

### SUMMARY OF THE INVENTION

The invention comprises an oral Omega-3-fatty acid-rich oil emulsion composition for use in management of treatment of a disorder to overcome the same. The Omega-3-fatty acid-rich oil may be microalge oil containing 40% DHA and the emulsion comprising DHA 50-100 mg/ml.

It is an embodiment of this invention that the said oral Omega-3-fatty acid-rich oil emulsion is made from ingredients all of which are natural and biocompatible ingredients, compatible for administration in Children including neonates

In one embodiment, this oral Omega-3-fatty acid-rich oil emulsion composition has the dispersed phase is in nanometric size range.

Invention is also claimed wherein the oral Omega-3-fatty acid-rich oil emulsion composition has stability at room temperature (about 30° C.) and refrigerated one (2-8° C.).

In another embodiment, the oral Omega-3-fatty acid-rich oil emulsion composition comprises Vitamins and Minerals in therapeutically effective amount.

The disorder treated by the oral Omega-3 fatty acid-rich oil emulsion comprises, without limitation, pre-term birth disorder in pregnant women, cognitive disorders in children and cardiovascular disorders.

The pre-term birth disorder in pregnant women is overcome by achieving normal delivery, cognitive disorders in children is overcome by improvement in their cognitive ability, and a cardiovascular disorder is overcome by return to healthy condition the oral Omega-3 fatty acid-rich oil emulsion of the instant invention.

In another embodiment this invention comprises in oral Omega-3 fatty-acid-rich oil thixotropic emulsion in nanomeric size range that has better absorption. The oral Omega-3-fatty-acid-rich oil may be as a thixotropic emulsion in nanometric size range having higher surface area and absorption. The oral Omega-3-fatty-acid-rich oil thixotropic emulsion comprises natural emulsifier and their derivatives and bio surfactants alone or in combination with vitamins, minerals, Generally Regarded As safe (GRAS) natural ingredients. The natural emulsifier comprise, without limitation, one or more of natural gums, clays, polymers etc.; additives comprising, without limitation, one or more selected from the group rheology modifiers, anti-oxidants, preservatives, stabilizers, sweetening and flavoring agents.

This invention also comprises an oral Omega-3 fatty-acid-rich oil emulsion composition having dispersed phase having nanometric size comprising natural emulsifier and their derivative surfactants alone or in combination with vitamins, minerals, Generally Regarded As safe (GRAS) natural ingredients.

This invention also embodies a High Performance Liquid Chromatography (HPLC) method for assay of omega-3 fatty acids. The method comprises steps of separately injecting blank, standard solutions-1, standard solutions-2 and sample solution into the chromatograph, recording the chromatograms and measuring the peak responses for Docosahexaenoic acid (DHA). The blank injected is in a single replicate, standard solutions-1 injected are in five replicates, standard solutions-2 injected are in two replicates, and sample solution injected is in a single replicate, the column used is Thermo Syncronis C18 (250×4.6 mm)—5 μm or Equivalent, pump mode is isocratic, flow rate is 1.0 ml/min, detection is at UV, 210 nm, injection volume is 20 μl, column oven temperature is 45° C. and run time is 20 minutes. The solution-1 comprises DHA working standard, the solution-2 comprises DHA test solution. The sample solution comprises known quantity of DHA-rich algal oil sonicated for a period of time with n-Heptane in around bottom flask, Methanolic Sodium hydroxide solution is added to the same and refluxed for 10 minutes with a stirrer, cooled in ice bath without removing the round bottom flask, slowly and Boron Triflouride Methanol Complex Solution is cautiously added, the solution is refluxing further with magnetic with stirrer, cooled in ice bath without removing the round bottom flask, slowly with cautiously adding n-heptane and refluxing, cooling the mixture and removing the round bottom flask, adding saturated Sodium chloride solution, shaking well and transferring the contents to a centrifuge tube, centrifuging with low speed, diluting upper Heptane layer with Isopropyl Alcohol and mixing the same, further diluting this solution with Methanol and mixing the same.

The natural emulsifiers comprise one or more selected from the group consisting of (i) Vitamin E TPGS (d-α-Tocopheryl polyethylene glycol 1000 succinate); (ii) Phospholipids comprise one or more selected from the group consisting of soya- and egg phosphatidylcholine, distearyl phosphatidylcholine, phosphatidylethanolamine and phosphatidylserine; gums comprise one or more selected from the group consisting of gum acacia, guar gum, xanthan gum, and gum targacanth; polymers comprise, one or more selected from the group consisting of pectin, gelatin and alginate; emulsion stabilizers comprise one or more selected from the group consisting of Xanthan gum, guar gum, gum acacia, Bentonite, glycerol and mixture thereof.

The oral Omega-3-fatty acid-rich oil comprise of one or more selected from the group consisting of microalgae oil, fish oil or flaxseeds oil. Antioxidants comprise one or more selected from the group consisting of butylated hydroxytoluene, rosemary oil, sodium ascorbate, Vitablend (consisting of Vitamin E and ascorbyl palmitate), sodium metabisulphite, ascorbyl palmitate and Vitamin E; vitamins comprise one or more selected from the group consisting of oil soluble vitamin A, vitamin D, vitamin E, vitamin K; water soluble Vitamin B1, Vitamin B2, Vitamin B3, Vitamin B5, Vitamin B6, Vitamin B12, folic acid and vitamin C. Minerals include zinc, copper, magnesium, potassium, calcium as calcium phosphate or calcium carbonate, iron and β-carotene etc.; buffers comprise one or more selected from the group consisting of sodium citrate sodium carbonate and phosphate buffer flavoring agent comprise one or more flavors selected from the group consisting of orange, strawberry, raspberry, mango, peach, vanilla, lime flavors; sweetening agents comprise one or more selected from the group consisting Sorbitol, xylitol, mannitol, Sucralose, Stevia, Aspartame, Neotame, Acesulfame K and mixtures thereof; preservative comprises rosemary extract, sodium benzoate, sodium azide, Methyl and propyl Paraben.

This invention also discloses a process of making oral Omega-3-fatty acid-rich oil emulsion comprising steps of: (a) making an oil phase of omega-3-fatty acid-rich microalgae oil by mixing a natural emulsifier, a blend of vitamins comprising an anti-oxidant with DHA-rich microalgae oil in a manufacturing tank with stirrer at room temperature, (b) making an aqueous phase in a tank with stirrer comprising steps of: (i) soaking a gum in purified water for a period of time required for dissolution, (ii) dissolving Vitamin E TPGS (d-α-Tocopheryl polyethylene glycol 1000 succinate) in another vessel under mechanical stirring, and other water soluble ingredients comprising a preservative and a high intensity sweetener were mixed with this solution, (iii) thereafter, both gum and Vitamin E and Ascorbyl Palmitate solutions were mixed under mechanical stirring for a period of time required to form uniform mixture, (iv) thereafter, oil phase was added to the aqueous phase under mechanical stirring maintaining both phases at room temperature, (v) adding flavoring agent, and continuing stirring for a further period of time.

The process of making oral Omega-3-fatty acid-rich oil emulsion according to claim 17, wherein, the natural emulsifier comprises Soya phosphatidyl choline, the oil phase comprises a mix of: (1) omega-3-fatty acid-rich microalgae oil 12.5-25% w/v, (II) Anti-oxidant mix (Vitablend™) 0.05 to 0.5%, (Ill) butylated hydroxytoluene 0.1%, (IV) DHA-rich microalgae oil 12.5-25% w/v, (V) the Manufacturing tank is stainless steel Jacketed, (VI) stirring is done at 40-50° C. with stirring speed of 100-300 RPM, (VII) the gum is Xanthan gum at 0.3-1.5% w/v), (VIII) soaking was done in purified water at 40-50° C. for 1-5 h period, (IX) Vitamin E TPGS (d-α-Tocopheryl polyethylene glycol 1000 succinate) is added at 1-5% w/v), mechanical stirring is done at 1000-1500 RPM at 1000-1500 RPM, preservative is sodium benzoate 0.02-0.1% w/v, (X) high intensity sweetener is Sucralose 0.1-0.5% w/v), (XI) uniform mixture is done by stirring and mixing of both gum and Vitamin E TPGS solutions is done under mechanical stirring at 1000-1500 RPM at 40-50° C. for 30-60 minutes, (XII) finally, adding the oil phase was to the aqueous phase under mechanical stirring is done at 1000-1500 rpm, maintaining both phases at 40-50° C., (XIII) flavor being added is orange oil at 0.5% w/v to 1.0% w/v and stirring continued at 1-2 hrs.

### DETAILED DESCRIPTION OF THE INVENTION

The inventors of the present invention found that the need to develop stable and therapeutically effective formulation containing O3FA moieties can be fulfilled by incorporation of O3FA-rich oil into a colloidal/disperse system having nanometric size for effective absorption across the GI tract. In one embodiment this invention comprises an emulsion wherein strength of DHA is 50-100 mg/ml with normal microalge oil (contain 40% DHA).

In one embodiment of this invention that the emulsion comprises vitamins and minerals in therapeutically effective amount.

Still further, the delivery system comprises stabilizer/s, emulsifier/s, antioxidant/s, sweetening, flavoring agent/s, and vehicles.

It is also an embodiment of this invention that only natural surfactants are used. No synthetic surfactants have been used, so the emulsion composition is good for administration in Children including neonates In a further embodiment of this invention, the dispersed phase of the emulsion is in a nanometric range. This makes the emulsion better for absorption of DHA.

In a further embodiment, the emulsion of the instant invention has good stability at room temperature (about 30° C. and also at refrigerated temperature (about 2-8° C.).

Further, the method of preparation of the emulsion is very simple and cost effective as compared to prior art methods of making emulsion of the DHA-rich oils, including micro algae oil having 40% DHA.

O3FA-rich oil is preferably used from microalgae but may also include other O3FA-rich oil sources such as fish oil and other sources of O3FA.

Examples for emulsifiers include Vitamin E TPGS (d-α-Tocopheryl polyethylene glycol 1000 succinate), phospholipids but not limited to, soya- and egg phosphatidylcholine, distearyl phosphatidylcholine, phosphatidylethanolamine, phosphatidylserine, gum acacia, guar gum, xanthan gum, targacanth, pectin, gelatin, alginate, bentonite. The emulsifier can be defined for the purpose of this invention as any molecule which possesses a Hydrophilic Lipophilic Balance (HLB) value between 4-18.

Emulsion stabilizers can function as thickening agents, which arrest the coalescences of oil droplets when used alone and when used in combination with rheological modifiers. Emulsion stabilizers include, but are not limited to, Xanthan gum, guar gum, gum acacia, Bentonite, glycerol and mixture thereof.

Antioxidants that prevent the autoxidation of poly unsaturated fatty acids include rosemary oil, sodium ascorbate, Vitablend, sodium metabisulphite, ascorbyl palmitate, Vitamin E etc.

Vitamins include, but are not limited to, oil soluble vitamin A, vitamin D, vitamin E, vitamin K; water soluble Vitamin B1, Vitamin B2, Vitamin B3, Vitamin B5, Vitamin B6, Vitamin B12, folic acid and Vitamin C. Minerals include zinc, copper, magnesium, potassium, calcium as calcium phosphate or calcium carbonate, iron and β-carotene etc.

Buffers include, but are not limited to, sodium citrate, sodium carbonate were used to maintain the pH of emulsion formulations.

Sweetening and flavoring agents increase the palatability of oral emulsion, they include, but are not limited to orange, strawberry, raspberry, mango, peach, vanilla, lime flavors and Sorbitol, xylitol, mannitol, Sucralose, Stevia, Aspartame, Neotame, Acesulfame K. etc. as sweeteners used alone and mixtures thereof.

The stability of oral O3FA-rich oil emulsion was assessed at different storage temperature and times points (0, 30, 60, 90 and 180 days) by assessing parameters DHA content, specific gravity, pH, peroxide value, rheological studies, peroxide and rancimat analysis

and therapeutic effectiveness of oral O3FA-rich oil emulsion was assessed by rheological studies, peroxide and rancimat analysis, in vivo cognitive animal models and ex vivo Caco2 and Fr2 cell line studies.

Information on enhanced storage shelf life and therapeutic effectiveness of the oral O3FA-rich oil emulsion is not available in prior art. The benefits of O3FA-rich oil emulsion formulation are augmented in this invention by making an oral composition of microalgae oil emulsion that has enhanced storage and oxidative stability. The emulsion of this invention is developed especially in treatment of pre-term birth disorder in pregnant women and cognitive disorders in children removed. The emulsion of this invention has shown to be effective in enhancing O3FA blood plasma levels over prior art compositions.

This invention discloses a composition of O3FA-rich oil emulsion that is stable in its physical properties and also has oxidative stability. This invention also discloses a process to prepare pharmaceutically acceptable and stable O3FA-rich oil emulsion formulations including solid self nano emulsifying drug delivery system, conventional emulsion, miceller dispersion alone and in combination of vitamins and minerals with GRAS (Generally regarded as safe) certified ingredients. The composition of O3FA-rich oil emulsion contains different natural emulsifiers such as natural gums and their derivatives and bio surfactants alone or in combinations. The composition also contains additives in optimized amount such as rheology modifiers, anti-oxidants, preservatives, stabilizers, sweetening and flavoring agents etc. Effect of type and amount of emulsifier, stabilizers and antioxidants on oxidative stability, storage shelf life, rheology and toxicity profile was studied by peroxide value, rheological analysis, cell line and animal studies, respectively. The resulted emulsion has enhanced storage shelf life and is resistant to oxidation and coalescences. The treatment of omega 3 fatty acid deficiency in humans or animals and for reducing risk in humans associated with pre-term birth or cognitive disorder or for cardiovascular health using developed formulation is disclosed. Further, new simple, accurate and precise HPLC method for assay of major omega 3 fatty acids has been disclosed in present invention.

This invention also embodies a novel High-performance Liquid Chromatography (HPLC) method of analysis of DHA content in samples. Current Association of Official Agricultural Chemists (AOAC) method is Gas Chromatography (GC) with Flame Ionization Detector (FID).

Related standard deviation of method of analysis with GC is 5% vs the same for inventive HPLC method is less than 2%. GC method reproducibility is less, the inventive HPLC method reproducibility is very good. Stability of sample solution is good in the inventive HPLC method, poor in GC.

In the following are provided illustrative examples of the compositions of this invention and investigations carried on them. The Examples are only illustrative and do not limit the scope of the claims that would be obvious to a person skilled in the art and that would be apparent as equivalent to a person skilled in the art.

### BRIEF DESCRIPTION OF FIGURES AND LEGENDS

FIG. 1: Illustrates the Transmission electron micrograph (TEM) of Emulsion formulation.

FIG. 2: Globule size analysis of emulsion formulation using Dynamic light scattering method.

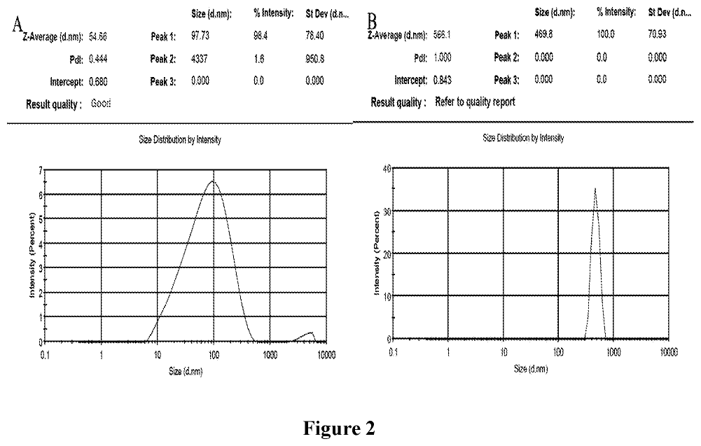

FIG. 3: Illustrates the rheological analysis of O3FA-rich oil formulation (A) at constant shear rate at 40 s⁻¹ (B) at variable shear rate 0-60 s⁻¹ (C) thixotropic analysis of O3FA-rich oil emulsion.

FIG. 4: Illustrates the peroxide value of O3FA-rich oil emulsion A) at real time condition B) at refrigerated conditions.

FIG. 5: Illustrates the induction time at which secondary oxidation products are formed in Rancimat analysis in emulsion (induction time 2.57 h) in comparison to pure oil (induction time 0.45 h), indicating the higher oxidative stability of emulsion.

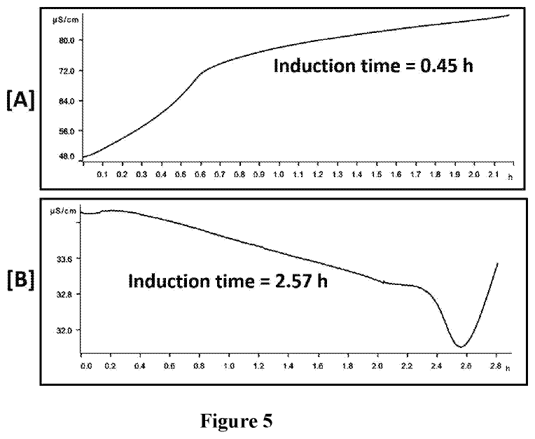

FIG. 6: Illustrates the biosafety of emulsion formulation in MTT assay against A) Caco-2 cell line B) Fr2 cell line.

FIG. 7: Illustrates A) Paracellular permeability B) TEER measurement of emulsion formulation. (Both parameter are the measures of changes in the tight junctional and paracellular spacing of gastrointestinal cells).

FIG. 8: Permeability of DHA from DHA emulsion formulation in comparison to DHA oil.

FIG. 9: Pharmacokinetic of optimized DHA emulsion formulation in comparison to DHA oil by measuring DHA content in phospholipids.

FIG. 10: HPLC chromatogram for method developed and validated for estimation of DHA.

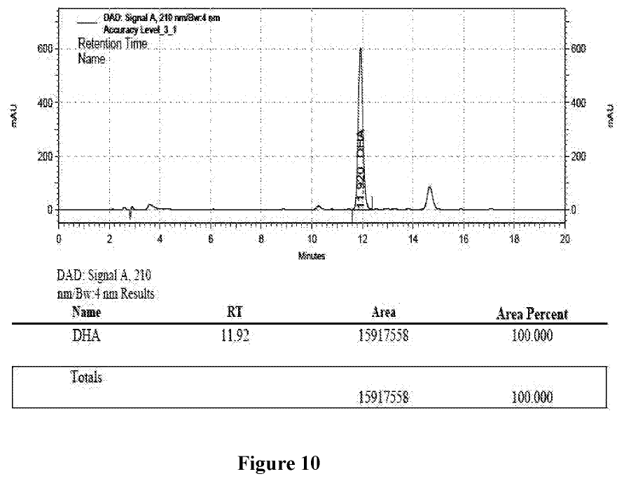

FIG. 11: Process flow diagram for commercial production of DHA emulsion.

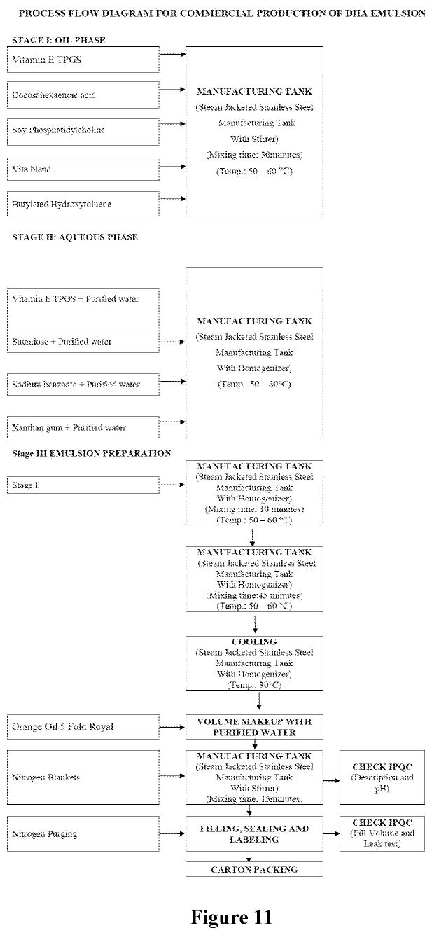

### EXAMPLE 1

Preparation of a O3FA-Rich Oil Emulsion Formulations

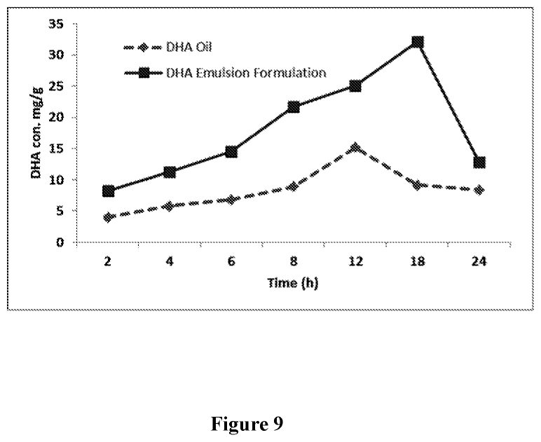

Emulsions were prepared using O3FA-rich microalgae oil making 5-25% of the total content of emulsion. In first step, oil phase was prepared by mixing 0.4% w/v of soya phosphatidyl choline, 0.05% of Vitablend, 0.1% of butylated hydroxytoluene with 12.5-25% w/v of DHA-rich microalgae oil in stainless steel Jacketed Manufacturing tank with stirrer at 40° C. with stirring speed of 100 RPM. In second step, aqueous phase was prepared in stainless steel Jacketed Manufacturing tank with stirrer. Xanthan gum (0.4% w/v) was soaked in purified water at 40° C. for 2-3 h period. In another vessel, Vitamin E TPGS (2% w/v) was dissolved under mechanical stirring at 1000-1200 RPM at 40° C. and other water soluble ingredients sodium benzoate (0.05% w/v) and Sucralose (0.1% w/v) were mixed with this solution. Then both gum and Vitamin E TPGS solutions were mixed under mechanical stirring at 1000-1200 RPM at 40° C. for 30 minutes to form uniform mixture. Finally, oil phase was added to the aqueous phase under mechanical stirring at 1000-1200 rpm, maintaining both phases at 40° C. Stirring was continued for 0.5-1.0 hr after addition of orange oil (0.5% w/v) as flavoring agent. Process flow diagram for commercial production of DHA emulsion in strength of 50-100 mg/ml is shown in FIG. 9.

TABLE 1
Composition of O3FA-rich oil emulsion
formulation in strength 50 mg/ml
(prototype 1)

| | | |
|---|---|---|
| Ingredients | Quantity for 100 ml | % w/v |

OIL PHASE

| | | |
|---|---|---|
| DHA oil (40%) | 12.500 g | 12.500% |
| Soy Phosphatidylcholine | 0.400 g | 0.400% |
| Ascorbyl Palmitate plus Vitamin E | 0.050 g | 0.050% |
| Butylated Hydroxytoluene | 0.100 g | 0.100% |
| Orange 5 Fold Royal Oil Flavour | 0.500 g | 0.500% |

WATER PHASE

| | | |
|---|---|---|
| Xanthan gum | 0.400 g | 0.540% |
| D-α-Tocopheryl polyethylene | 2.000 g | 2.000% |
| glycol 1000 | | |
| succinate (Vitamin E TPGS) | | |
| Sodium Benzoate | 0.050 g | 0.050% |
| Sucralose | 0.100 g | 0.100% |
| Purified water | QS for 100 ml | 71.4% |

TABLE 2
Composition of O3FA-rich oil emulsion
formulation 75 mg/ml (prototype 2)

| | | |
|---|---|---|
| Ingredients | Quantity for 100 ml | % w/v |

OIL PHASE

| | | |
|---|---|---|
| DHA oil (40%) | 18.750 g | 18.750% |
| Soy Phosphatidylcholine | 0.400 g | 0.400% |
| Ascorbyl Palmitate plus Vitamin E | 0.050 g | 0.050% |
| Butylated Hydroxytoluene | 0.100 g | 0.100% |
| Orange 5 Fold Royal Oil Flavour | 0.500 g | 0.500% |

WATER PHASE

| | | |
|---|---|---|
| Xanthan gum | 0.400 g | 0.400% |
| D-α-Tocopheryl polyethylene | 2.000 g | 2.000% |
| glycol 1000 | | |
| succinate (Vitamin E TPGS) | | |
| Sodium Benzoate | 0.050 g | 0.050% |
| Sucralose | 0.100 g | 0.100% |
| Purified water | QS for 100 ml | 77.650% |

TABLE 3
Composition of O3FA-rich oil emulsion
formulation 100 mg/ml (prototype 4)

| | | |
|---|---|---|
| Ingredients | Quantity for 100 ml | % w/v |

OIL PHASE

| | | |
|---|---|---|
| DHA oil (40%) | 18.750 g | 25.000% |
| Soy Phosphatidylcholine | 0.400 g | 0.400% |
| Ascorbyl Palmitate plus Vitamin E | 0.050 g | 0.050% |
| Butylated Hydroxytoluene | 0.100 g | 0.100% |
| Orange 5 Fold Royal Oil Flavour | 0.500 g | 0.500% |

WATER PHASE

| | | |
|---|---|---|
| Xanthan gum | 0.400 g | 0.400% |
| D-α-Tocopheryl polyethylene | 2.000 g | 2.000% |
| glycol 1000 | | |
| succinate (Vitamin E TPGS) | | |
| Sodium Benzoate | 0.050 g | 0.050% |
| Sucralose | 0.100 g | 0.100% |
| Purified water | QS for 100 ml | 71.400% |

TABLE 4
Composition of O3FA-rich oil emulsion formulation with Vitamins
using combination of natural emulsifiers (Prototype 4)

| | | |
|---|---|---|
| Ingredients | Quantity for 100 ml | % w/v |

OIL PHASE

| | | |
|---|---|---|
| DHA oil (40%) | 12.750 g | 25.000% |
| Soy Phosphatidylcholine | 0.400 g | 0.400% |
| Ascorbyl Palmitate plus Vitamin E | 0.050 g | 0.050% |
| Butylated Hydroxytoluene | 0.100 g | 0.100% |
| Orange 5 Fold Royal Oil Flavour | 0.500 g | 0.500% |

WATER PHASE

| | | |
|---|---|---|
| Xanthan gum | 0.500 g | 0.400% |
| Pectin | 0.100 g | 0.100% |
| Bentonite | 0.500 g | 0.500% |
| D-α-Tocopheryl polyethylene | 2.000 g | 2.000% |
| glycol 1000 | | |
| succinate (Vitamin E TPGS) | | |
| Sodium Benzoate | 0.050 g | 0.050% |
| Sucralose | 0.100 g | 0.100% |
| Purified water | QS for 100 ml | 69.800% |

### EXAMPLE 2

Preparation of O3FA-Rich Oil Emulsion Fortified with Vitamins and Minerals

Method of making the basic emulsion was as provided in example 1, to which Vitamins and minerals and folic acid were added. The composition of this product has been given in Table 5 and 6.

TABLE 5
Composition of O3FA-rich oil emulsion formulation
fortified with Vitamins and Minerals

| | | |
|---|---|---|
| Ingredients | Quantity for 100 ml | % w/v |

OIL PHASE

| | | |
|---|---|---|
| DHA oil (40%) | 12.500 g | 12.500% |
| Soy Phosphatidylcholine | 0.400 g | 0.400% |
| Ascorbyl Palmitate plus Vitamin E | 0.050 g | 0.050% |
| Butylated Hydroxytoluene | 0.100 g | 0.100% |
| Orange 5 Fold Royal Oil Flavour | 0.500 g | 0.500% |
| Retinal (as vitamin A palmitate) | 0.040 g | 0.040% |
| Ergocalciferol solution | 0.004 g | 0.004% |

WATER PHASE

| | | |
|---|---|---|
| Xanthan gum | 0.400 g | 0.400% |
| Thiamine hydrochloride | 0.040 g | 0.040% |
| Nicotinamide | 0.800 g | 0.800% |
| Ascorbic Acid | 4.000 g | 4.000% |
| Zinc (as zinc gluconate) | 0.697 g | 0.697% |
| Mangnese (as manganese gluconate) | 0.810 g | 0.810 g |
| D-α-Tocopheryl polyethylene | 2.000 g | 2.000% |
| glycol 1000 | | |
| succinate (Vitamin E TPGS) | | |
| Sodium Benzoate | 0.050 g | 0.050% |
| Sucralose | 0.100 g | 0.100% |
| Purified water | QS for 100 ml | 77.509% |

TABLE 6
Composition of O3FA-rich oil emulsion formulation
fortified with Vitamins and Minerals

| | | |
|---|---|---|
| Ingredients | Quantity for 100 ml | % w/v |

OIL PHASE

| | | |
|---|---|---|
| DHA oil (40%) | 12.500 g | 12.500% |
| Soy Phosphatidylcholine | 0.400 g | 0.400% |
| Ascorbyl Palmitate plus Vitamin E | 0.050 g | 0.050% |
| Butylated Hydroxytoluene | 0.100 g | 0.100% |
| Orange 5 Fold Royal Oil Flavour | 0.500 g | 0.500% |
| Retinal (as vitamin A palmitate) | 0.040 g | 0.040% |
| Ergocalciferol solution | 0.004 g | 0.004% |

WATER PHASE

| | | |
|---|---|---|
| Xanthan gum | 0.400 g | 0.400% |
| Pectin | 0.100 g | 0.100% |
| Bentonite | 0.500 g | 0.500% |
| Thiamine hydrochloride | 0.040 g | 0.040% |
| Nicotinamide | 0.800 g | 0.800% |
| Ascorbic Acid | 4.000 g | 4.000% |
| Zinc (as zinc gluconate) | 0.697 g | 0.697% |
| Mangnese (as manganese gluconate) | 0.810 g | 0.810% |
| Folic acid | 0.010 g | 0.010% |
| D-α-Tocopheryl polyethylene | 2.000 g | 2.000% |
| glycol 1000 | | |
| succinate (Vitamin E TPGS) | | |
| Sodium Benzoate | 0.050 g | 0.050% |
| Sucralose | 0.100 g | 0.100% |
| Purified water | QS for 100 ml | 76.9% |

### EXAMPLE 3

In Vitro Characterization for Quality Control Tests

Following are the properties/characteristics studied/determined and the results are provided in Table 7 below.

Globule Size and Zeta Potential Analysis

Globule size, size distribution and zeta potential of emulsion was measured on O3FA-rich oil emulsion prepared in Examples using particle size analyzer (Malvern Zetasizer Nano ZS90, UK) by 100 times diluting the sample with triple distilled water. In addition to average particle size the intensity distribution of the particles and polydispersity index (PDI) which is a measure of uniformity in size distribution were also measured.

pH of Emulsion Formulations

O3FA-rich oil emulsions were developed for the oral administration; therefore the pH of the emulsion should be within the acceptable range required for the oral administration i.e. in between pH 5-8. The pH of the prepared emulsion formulations was determined using pH meter (Toshcon CL-54).

Transmission Electron Microscopy (TEM) Analysis

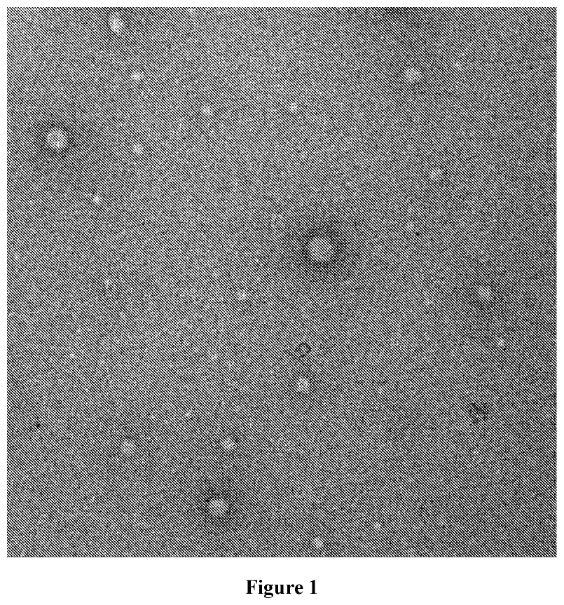

The size and shape of dispersed phase in the emulsion system was observed by Transmission electron microscopy (TEM), (HRTEM, JEM 2100, JEOL, Japan) operated at an accelerating voltage of 200 kV with beam current of 100 μA. The sample was diluted in ratio of 1:100 with triple distilled water and then a drop of diluted emulsion was placed directly onto a carbon-coated copper grid. The grid was air dried and was observed under transmission electron microscope. Results are provided in FIG. 1.

Thermodynamic Stability Study

The thermodynamic stability of the O3FA-rich oil emulsion formulations was determined by storing the emulsion samples at different temperature conditions 4±1° C. and 45±1° C. for 24 h storage at each temperature. Finally, after cooling and heating cycle sample was subjected to centrifugation stress at 3000 rpm for 10 min and the extent of any oil separation was monitored.

Viscosity

The viscosity of the prepared emulsion was determined by Brookfield viscometer using spindle no. 5 at constant shear rate of 40 rpm/min.

Dispersibility Test and Robustness to Dilution

Five ml of the prepared formulations were added to 500 ml of distilled water in USP Type II Dissolution apparatus (Lab India DS 8000) at 37±0.5° C. and 50 rpm. The formulations were visually examined for water dispersibility immediately after addition to the vessel using following grading system: Grade A—Rapidly dispersing milky emulsion (<1 minutes); Grade B—Moderate dispersing milky emulsion (>2 minutes); Grade C: Slow dispersion milky emulsion (>5 minutes) with appearance of oil. Robustness to dilution is an important parameter for emulsion to ensure that the prepared emulsion has similar properties at different dilutions after oral administration. 1 ml of each emulsion was diluted to 100 and 1000 times with distilled water and 0.1 N HCl. The diluted emulsions were observed for 24 h in order to determine the separation of oily phase at higher dilutions and globule size. Robustness to dilution is an important parameter to understand the behaviour of emulsion globules under in vivo conditions. Separation of oily phase leads to poor intestinal absorption. The results obtained are provided in Table 7.

TABLE 7
In vitro characterization of O3FA-rich oil emulsion formulations

| | | O3FA emulsion |
|---|---|---|
| | O3FA emulsion | formulation fortified with |
| Characteristic | formulation | Vitamins and Minerals |
| Particle size (nm) | 54.6 nm | 146 nm |
| after 10 fold | | |
| dilution with water | | |
| Particle size (nm) | 566.1 nm | 612 nm |
| after 100 fold | | |
| dilution with water | | |
| Zeta potential | −17.0 mV | −17.2 mV |
| pH | 6.52 | 5.74 |
| Thermodynamic | Stable and | Stable and |
| stability | No creaming and | No creaming and |
| | no cracking. | no cracking |
| Heat cooling cycle | Pass | Pass |
| Shape | Globular | Globular |
| Viscosity (Brookfield | 410 cp | 990 cp |
| Viscometer | | |
| spindle no. 5, 40 | | |
| RPM at RT) | | |
| Dilution integrity | | |
| 0.1 N HCl | Good (No precipitation) | Good (No precipitation) |
| PBS 6.8 | Good (No precipitation) | Good (No precipitation) |
| PBS 7.4 | Good (No precipitation) | Good (No precipitation) |
| Water | Good (No precipitation) | Good (No precipitation) |
| Dispersibility grade | Grade A (Rapidly | Grade A (Rapidly |
| | dispersing milky | dispersing milky |
| | emulsion (<1 minutes) | emulsion (<1 minutes) |

### EXAMPLE 4

Rheological Analysis

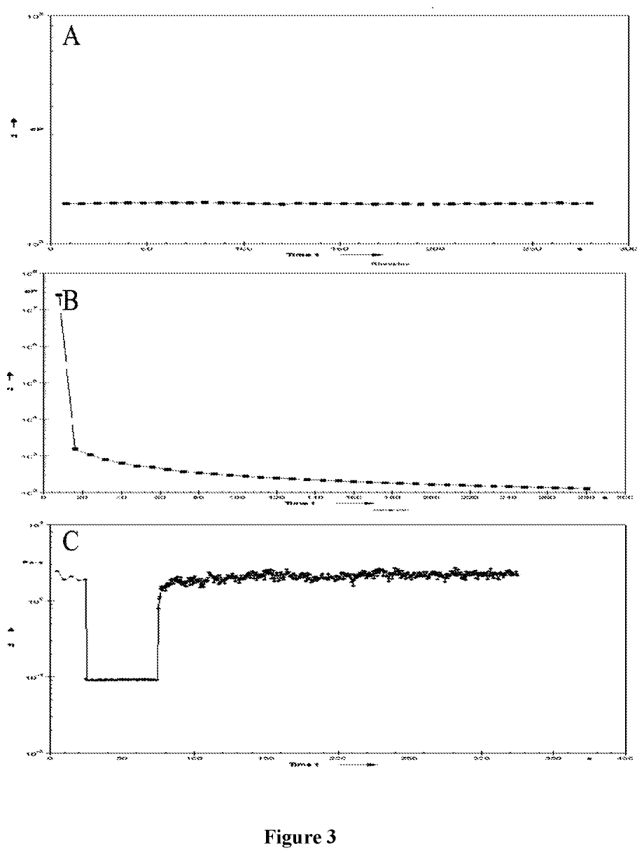

Rheological properties are important parameters for accessing the physical stability and thixotropic behavior of emulsion when subjected to different shear rates and stress. Viscosity and thixotropic profiles of O3FA emulsion was determined by using rheometer (Rheolab QC, Anton paar, Germany). Viscosities were determined at a constant shear rate (40 s⁻¹) and varying shear rate (0-60 s⁻¹) at 25° C. temperature conditions. The thixotropic behavior of O3FA emulsion was determined at shear rate of 80 s⁻¹ in order to evaluate the strength recovery ratio of the prepared emulsion after application and removal of shear stress. Results obtained are given in Table 8 and FIG. 3.

TABLE 8
Rheological behavior of emulsion formulation.

| S | Apparent Viscosity at | Thixotrophy | |
|---|---|---|---|
| no. | constant shear rate (40 s⁻¹) | (% Recovery) | Breakdown |
| 1 | 150- 850cp | 97.0-100.25 | No |

### EXAMPLE 5

Peroxide Value Estimation

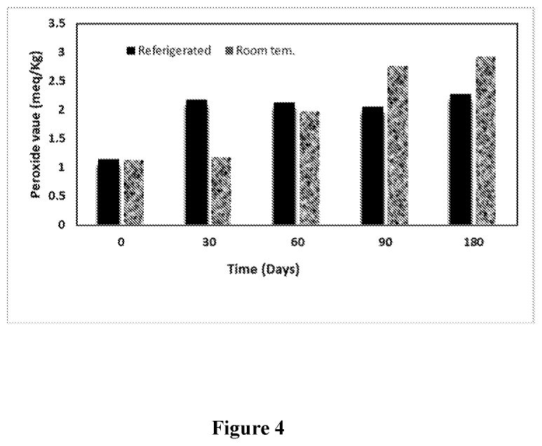

Peroxides are the primary products of oxidation formed during oxidation of oils and lipids. They were measured according to the official method followed by Association of Analytical Communities (AOAC) at time interval of 0, 30, 60 and 90 days. During this study the samples were stored under accelerated storage conditions and refrigerated condition. Acetic acid-chloroform mixture (30 mL) in the ratio 3:2 was added to the 5 ml of emulsion in conical flask and the content was properly shaken. 0.5 mL saturated solution of potassium iodide (PI) was added and conical flask was placed in dark with occasional shaking for 1 min. 0.5 mL of 1% w/v freshly prepared starch solution was added and titration was done with 0.01 N sodium thiosulphate with vigorous shaking until blue color was disappeared. The peroxide value was calculated by using the following equation. Results obtained are given in FIG. 4.

$$Peroxide value ( meq ) = \frac{( S - B ) \times N ( {Na}_{2} {S}_{2} {O}_{3} )}{Weight of sample} \times 1000 \\ S = titration volume of sample \\ B = titration volume of blank$$

### EXAMPLE 6

Rancimat Analysis

Deterioration of taste and odor (rancidity) due to oxidation is slow process at ambient temperature; rancimat method accelerates the natural autooxidation process and determine the oxidative stability of the products with time. It was determined using 892 Professional Rancimat apparatus from Metrohm, Herisau, Switzerland at temperature condition of 90° C. with air flow rate of 20 mL air/h. 3 ml of emulsion formulation was kept in reaction vessels which were attached with measuring vessel and air flow pipes. Secondary metabolites formed were transfer to measuring vessel with the air flow and change in the conductivity of measuring solution as inflection point was measured. Results are provided in FIG. 4.

### EXAMPLE 7

Stability Study

Accelerated stability testing is going on as per ICH (International Conference for Harmonization) guidelines by storing the O3FA-rich emulsion in sealed amber colored bottles at refrigerated condition (2 to 8° C.), and at accelerated conditions (40° C./75% RH), real time (25° C./75% RH) by using stability chamber and results are provided in Table 9.

TABLE 9
Results of stability studies under real time
and refrigeration storage conditions.

| Parameters | Initial | 1month | 2months | 3months | 6 months |
|---|---|---|---|---|---|
| | Storage Conditions: 25° C. ± 2° C./60% ± | | | | |
| | 5% RH (Room temperature) | | | | |
| Appearance | Light yellow | NC | NC | NC | NC |
| | coloured | | | | |
| | emulsion | | | | |
| | having | | | | |
| | sweet taste | | | | |
| | and pleasant | | | | |
| | odour. | | | | |
| Physical | Stable | Stable | Stable | Stable | Stable |
| Stability | | | | | |
| Viscosity | 410 | 410 | 430 | 460 | 520 |
| (cP) | | | | | |
| pH | 5.46 | 5.60 | 5.61 | 5.61 | 5.69 |
| Z-Average | 566.1 | 560.8 | 539.1 | 565.8 | 585.1 |
| (nm) after | | | | | |
| 100× dil | | | | | |
| Z-Average | 54.6 | 50.5 | 51.7 | 52.2 | 55.7 |
| (nm) after | | | | | |
| 10× dil | | | | | |
| Specific | 0.9934 | 0.9920 | 0.9932 | 0.9961 | 0.9978 |
| Gravity | | | | | |
| g/ml | | | | | |
| Thixotropy | 99.41 | 99.23 | 97.63 | 96.1 | 94.5 |
| (%) | | | | | |
| Peroxide | 1.13 | 1.88 | 1.98 | 2.77 | 2.92 |
| value | | | | | |
| (meq/Kg) | | | | | |
| Assay (% | 109.1 | 108.4 | 105.3 | 103.4 | 102.8 |
| DHA | | | | | |
| Content) | | | | | |
| | Refrigerated Storage Condition: 5° C. ± 3° C. | | | | |
| Appearance | Light yellow | NC | NC | NC | NC |
| | coloured | | | | |
| | emulsion | | | | |
| | having | | | | |
| | sweet taste | | | | |
| | and pleasant | | | | |
| | odour. | | | | |
| Physical | Stable | Stable | Stable | Stable | Stable |
| Stability | | | | | |
| Viscosity | 410 | 410 | 425 | 445 | 490 |
| (cP) | | | | | |
| pH | 5.46 | 5.65 | 5.65 | 5.69 | 5.72 |
| Z-Average | 562.5 | 556.4 | 565.8 | 539.1 | 546.9 |
| (nm) after | | | | | |
| 100× dil | | | | | |
| Z-Average | 49.5 | 42.7 | 52.2 | 55.7 | 55.5 |
| (nm) after | | | | | |
| 10× dil | | | | | |
| Specific | 0.9934 | 0.9967 | 0.9901 | 0.9886 | 0.9812 |
| gravity | | | | | |
| g/ml | | | | | |
| Thixotropy | 99.41 | 99.23 | 97.63 | 96.1 | 96.22 |
| (%) | | | | | |
| Peroxide | 1.13 | 2.17 | 2.12 | 2.05 | 2.27 |
| value | | | | | |
| (meq/Kg) | | | | | |
| Assay (% | 109.1 | 105.9 | 104.6 | 104 | 103.8 |
| DHA | | | | | |
| Content) | | | | | |

All value is expressed as mean ± SD (n = 3).
“*” represents the significant change in parameter where p < 0.05.

### EXAMPLE 8

Cell Cytotoxicity Assay

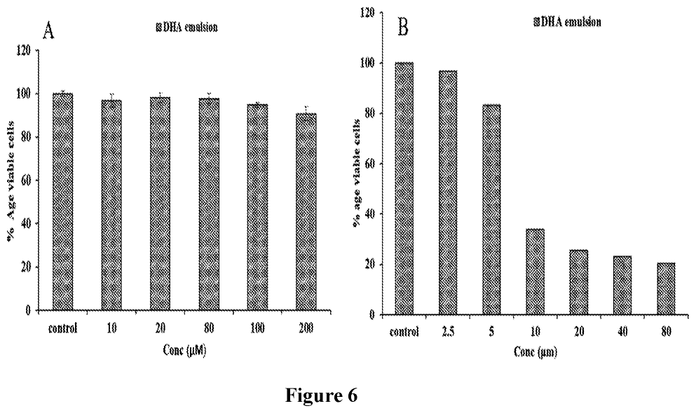

Cell viability assay was performed in Human colorectal (Caco2) and Normal Breast Epithelial (Fr2) cell lines with standard MTT (3-(4,5-Dimethylthiazol-2-yl)-2,S-diphenyltetrazolium bromide) method by using Human colorectal cell line (Caco2). Both the cell line were seeded in 96-well plate and Caco2 cell line was treated with 10, 20, 40, 80, 100 and 200 μM O3FA concentrations whereas Fr2 was treated with 25, 12.5, 6.25, 3.12, 1.56 and 0.78 mg/ml O3FA concentration present in emulsion formulations for 24 hour. After O3FA-rich oil treatment, media of each well was supplemented with 20 μL of MTT [3-(4,5-dimethylthaiazole-2-yl)-2,5-diphenyltetrazolium bromide] reagent and were further allowed to incubate for 4 h. Formed formazan crystals were solubilized by adding 200 μL of DMSO (Dimethyl Sulphoxide) into each well. In proportion of viable cells violet crystals were visible and absorbance was taken at 570 nm in microplate reader (Model: Omega fluostar, BMG Labtech Ltd., Germany). The results of % cell viability are summarized in FIG. 6. and it clearly indicated that the developed DHA emulsion formulation have more than 90% cell viability at all treated concentrations

### EXAMPLE 9

Transepithelial Electrical Resistance

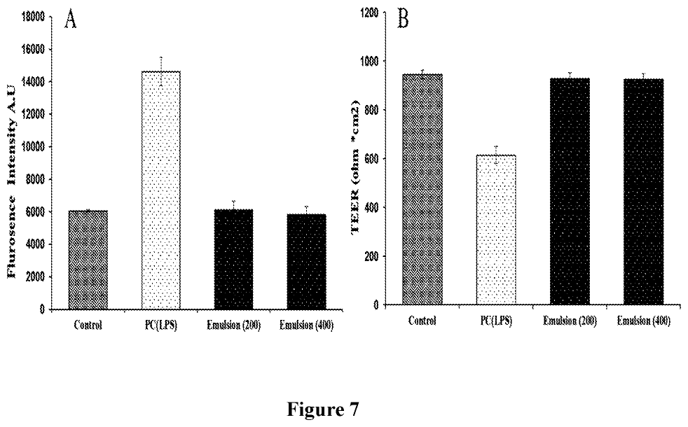

Human colorectal cell line, (Caco-2) were cultured in Dulbecco's modified Eagle's medium (DMEM) high Glucose supplemented with 10% FBS and 10% antibiotic-antimycotic in a humidified incubator with 5% CO₂ supply at 37° C. Cells were grown up to 60-70% confluency and harvested with trypsin-EDTA (0.25%) and seeded at a density of 2×10⁵ cells/mL onto polycarbonate membrane Transwell inserts with pore size of 0.4 μm. Cells were cultured for 14 days to reach differentiation, and growth media was refreshed every 2-3 days. Differentiated Caco-2 cells were treated with two concentrations of optimized formulation (200 μg/mL) and lipopolysaccharides (1 μg/mL), taken as a positive control. After incubation period of 48 h, TEER was measured using epithelial volt-ohm meter with a chopstick electrode (Millicell ERS-2, EMD Millipore, Billerica, Mass.) against control. Unit area resistance was calculated by dividing the resistance values with effective membrane area (4.52 cm²). Observed findings are shown in FIG. 7. Treatment with developed emulsion formulations and microalgae DHA oil did not show any significant change in TEER value in comparison to negative control, whereas as significant (p<0.001) decrease in TEER value in positive control (lipopolysaccharides) was observed (FIG. 7). Thus, confirming the developed formulations did not alter the integrity of the cellular barrier of Caco-2 cells

### EXAMPLE 10

Determination of Paracellular Permeability

After measurement of TEER value, treated Caco-2 cells were further analyzed for paracellular permeability by using fluorescein isothiocyanatedextran (FD, 4 kDa). FD was dissolved in phosphate buffer saline (PBS) to make concentration of 1 mg/mL. 0.2 mL of dye was added to the apical compartment of each insert, while 1.0 mL of PBS was added to the basolateral well.

The plate was covered with foil to prevent light inactivation of the fluorescent markers and placed in a shaker incubator at 37° C. at 150 rpm. After 12 h, 0.3 mL aliquots were taken from the basolateral chamber and fluorescence intensity was measured using a black 96 well plate in multiplate reader (Fluostar Omega, BMG Lab tech, Germany). Observed findings are shown in FIG. 7

Intestinal Permeation Measurement

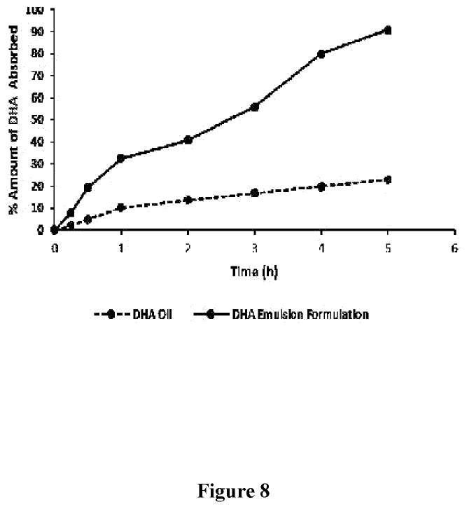

The study was performed goat ileum for developed emulsion formulation and DHA oil. With the Tyroid solution of pH 7.4, tissue was separated and cleaned. The thread was tied to one end of the ileum and placed in the student organ bath containing the Tyrode solution of pH 7.4. To prevent peristaltic muscle contractions, the liquid solution was packed into a bag and the weight of 1 g was attached to the unlined part of the lever. Aeration and bath temperatures around the ileum bag were established at 37±0.5° C. Samples were taken from the organ tube at various intervals of 0.25, 0.5, 1, 2, 4, and 5 h, and the DHA content was measured. Results are shown in FIG. 8.

Pharmacokinetic Study

Pharmacokinetic study was done for the period of 24 h with DHA emulsion formulation in comparison to DHA oil and 2.2 fold increases in the absorption of DHA was observed form the developed DHA formulation (FIG. 9). This increase of DHA in plasma was depicted from DHA content analysis by Gas Chromatography. The improved plasma levels of DHA observed during pharmacokinetic study resulted from the increased permeability of DHA from intestine, which was clearly demonstrated from the results of ex vivo intestinal permeability study.

### EXAMPLE 11

Development and Validation of HPLC Assay for Omega 3 Fatty Acids

New HPLC assay method has been developed with following chromatographic conditions have been optimized as specified in Table 10.

TABLE 10
Advantages of HPLC over GC for Analysis of DHA
content in DHA-rich oil Emulsion and Oil

| S.No. | Parameters | HPLC Method | GC Method |
|---|---|---|---|
| 1 | Type of | Its non-destructive | It is a destructive |
| | Method | method, If the | method, If the |
| | | sequence run not | sequence run not |
| | | completed due to | completed due to |
| | | errors like Instrument | errors like Instrument |
| | | error, power failure | error, power failure |
| | | etc. the same sample | etc. the same |
| | | solutions can be | sample solutions cannot |
| | | re-injected. | be re-injected. |
| 2 | Accuracy | More precise | Less precise and accurate |
| | and | and accurate. | comparing to HPLC. |
| | Precision | RSD limit: NMT 2.0%. | RSD limit: NMT 5.0%. |
| 3 | Analysis | 20 Min (RT 11.89 Min) | More comparing |
| | Time | | to HPLC |
| 4 | Solutions | Stable | Less stable |
| | stability | | |
| 5 | Mobile | Water:Methanol (950:50) | GC Gases (Nitrogen, |
| | Phase | (General and | Hydrogen and Zero |
| | | Regular solvents | Air). Handling of |
| | | used in Lab and less cost) | Gases is difficult. |
| 6 | For | HPLC Systems Generally | GC Instruments |
| | routine | readily available in | generally not |
| | analysis | Analytical laboratories | readily available |
| | | hence can be preferred. | in Analytical |
| | | | laboratories hence cannot |
| | | | prefer over HPLC. |
| 7. | Column | C18 (250 × 4.6 mm), | Dedicated column: |
| | | 5μ Thermo Synchronis | Fused silica |
| | | or other equivalent | capillary column |
| | | column, readily | (Cyanopropyl |
| | | available in | 90%, phenyl |
| | | Analytical Laboratory. | siloxane 10%). |
| | | Column cost is less | More cost comparing |
| | | comparing to | to HPLC columns. |
| | | GC columns. | |
| 8. | Handling | Handling is easy | Handling of gases is |
| | | comparing to GC. | difficult in GC analysis. |

Chromatographic Conditions:

| | |
|---|---|
| Column | : Thermo Syncronis C18 (250 × |
| | 4.6 mm), 5 μm or Equivalent |
| Pump mode | : Isocratic |
| Flow rate | : 1.0 ml/min |
| Detection | : UV, 210 nm |
| Injection volume | : 20 μl |
| Column oven temperature | : 45° C. |
| Run time | : 20 minutes |

Optimized method was found to accurately measure the DHA content in experimental protocols with high accuracy and validated for following parameters with RSD value of less than 2%. 1: Specificity and Identification, 1.1: Forced Degradation study; 2: Solution stability, 3: Linearity, 4: Accuracy, 5: Precision—5.1: System precision, 5.2: Method precision/Repeatability, 5.3: Intermediate precision/Ruggedness, 6: Range, 7: Robustness—7.1: Effect of Variation in flow rate, 7.2: Effect of Variation in Wavelength of detection, 7.3: Effect of Variation in Column oven temperature, 8: System suitability.

Preparation of Standard Solution:

Weigh accurately 250 mg of DHA working standard (625 mg of 40% DHA-rich microalgae oil) into a round bottom flask, add 10 mL n-Heptane by using pipette and sonicate for 15 minutes with intermittent shaking. Add 20 mL of 0.5N Methanolic Sodium hydroxide solution, connect to the condenser through Claisen adapter. Reflux the contents for 10 minutes with Magnetic Stirrer and cool for 5 minutes in ice bath without removing the round bottom flask. Slowly with cautiously add 20 mL of Boron Triflouride Methanol Complex Solution (13-15%) through the Claisen adapter and reflux for further 30 minutes with magnetic stirrer. Cool for 5 minutes in ice bath without removing the round bottom flask. Slowly with cautiously add 10 ml of n-heptane through Claisen adapter and reflux for further 5 minutes. Cool the mixture and remove the round bottom flask. Add 5 mL of saturated Sodium chloride solution, shake well and transfer the contents to a centrifuge tube. Centrifuge with low speed (500 RPM) for 5 minutes. Dilute 2 mL of upper Heptane layer to 50 mL with Isopropyl Alcohol and mix. Further dilute 5 mL of this solution to 50 mL with Methanol and mix.

Preparation of Sample Solution: (DHA-Rich Oil Emulsion)

Weigh the sample accurately equivalent to 250 mg of DHA-rich oil Emulsion into a round bottom flask, add 10 mL n-Heptane by using pipette and sonicate for 15 minutes with intermittent shaking. In illustrative work, DHA-rich microalgae oil was used; however, any other DHA-rich oil can be used. Add 20 mL of 0.5N Methanolic Sodium hydroxide solution, connect to the condenser through Claisen adapter. Reflux the contents for 10 minutes with Magnetic Stirrer and cool for 5 minutes in ice bath without removing the round bottom flask. Slowly with cautiously add 20 mL of Boron Triflouride Methanol Complex Solution (13-15%) through the Claisen adapter and reflux for further 30 minutes with magnetic stirrer. Cool for 5 minutes in ice bath without removing the round bottom flask. Slowly with cautiously add 10 ml of n-heptane through Claisen adapter and reflux for further 5 minutes. Cool the mixture and remove the round bottom flask. Add 5 mL of saturated Sodium chloride solution, shake well and transfer the contents to a centrifuge tube. Centrifuge with low speed (500 RPM) for 5 minutes. Dilute 2 mL of upper Heptane layer to 50 mL with Isopropyl Alcohol and mix. Further dilute 5 mL of this solution to 50 mL with Methanol and mix.

Sample Solution: (DHA-Rich Oil)

Weigh the sample accurately equivalent to 250 mg of DHA-rich algal oil into a round bottom flask, add 10 mL n-Heptane by using pipette and sonicate for 15 minutes with intermittent shaking. Add 20 mL of 0.5N Methanolic Sodium hydroxide solution, connect to the condenser through Claisen adapter. Reflux the contents for 10 minutes with Magnetic Stirrer and cool for 5 minutes in ice bath without removing the round bottom flask. Slowly with cautiously add 20 mL of Boron Triflouride Methanol Complex Solution (13-15%) through the Claisen adapter and reflux for further 30 minutes with magnetic stirrer. Cool for 5 minutes in ice bath without removing the round bottom flask. Slowly with cautiously add 10 ml of n-heptane through Claisen adapter and reflux for further 5 minutes. Cool the mixture and remove the round bottom flask. Add 5 mL of saturated Sodium chloride solution shake well and transfer the contents to a centrifuge tube. Centrifuge with low speed (500 RPM) for 5 minutes. Dilute 2 mL of upper Heptane layer to 50 mL with Isopropyl Alcohol and mix. Further dilute 5 mL of this solution to 50 mL with Methanol and mix.

Procedure:

Separately inject 20 μl of solvent blank (solvent system without analyte), standard solutions-1 (containing DHA in known amount) five replicates), standard solutions-2 (containing DHA sample for analysis (three replicates) and sample solution (single) into the chromatograph, record the chromatograms and measure the peak responses for DHA.

Retention Time of DHA peak is about 11.5 minutes.

System Suitability:

1) The column efficiency should be not less than 2000 theoretical plates for principal peaks.

2) The Theoretical plates should be not more than 2.0 for principal peaks.

3) The relative standard deviation for area response of principal peaks in five replicate injections of standard solution should be not more than 2.0%.

4) Asymmetry (Tailing factor) in each condition is not more than 2.0.

TABLE 11
Validation of developed HPLC method for analyzing DHA

| | | | Acceptance | | | |
|---|---|---|---|---|---|---|
| S. No. | Parameter | Experiment | criteria | Results | | |
| 1. | Specificity | Blank, | There should | There is no interference due to diluent and placebo | | |
| | | placebo, | not be any | at the retention time of DHA peak in standard run. | | |
| | | standard | interference | The identified impurities peaks are well separated | | |
| | | solution and | due to blank, | from DHA peak. | | |
| | | test solution. | placebo and | Sample name | Retention time (minutes) | |
| | | | palmetic acid | Standard solution | 11.87 | |
| | | | at the | Control sample | 11.87 | |
| | | | retention time | | | |
| | | | of DHA peak | | | |
| | | | Peak purity | DHA Peak | | |
| | | | factor should | Sample | Purity Factor | Result |
| | | | be within the | Standard solution | The purity factor | Pass |
| | | | calculated | | is within the | |
| | | | threshold limit | | calculated | |
| | | | for DHA peak | | threshold limit | |
| | | | in standard | Control test | The purity factor | Pass |
| | | | and control | solution | is within the | |
| | | | test solution | | calculated | |
| | | | | | threshold limit | |
| | | Assay of | Control | Sample ID | % Assay | |
| | | control sample | sample result | Control sample | 101.4 | |
| | | | should be | | | |
| | | | report | | | |
| 2. | | Forced | No peaks | DHA Peak | | |
| | | degradation | should be | Sample | Purity Factor | Result |
| | | Stressed | detected at | Control sample | The purity factor | Pass |
| | | sample | the retention | | is within the | |
| | | | time of DHA | | calculated | |
| | | | peak in the | | threshold limit | |
| | | | chromatogram | Acid Degradation | The purity factor | Pass |
| | | | of the diluent | | is within the | |
| | | | and placebo. | | calculated | |
| | | | Peak purity | | threshold limit | |
| | | | factor should | Base | The purity factor | Pass |
| | | | be within the | Degradation | is within the | |
| | | | calculated | | calculated | |
| | | | threshold limit | | threshold limit | |
| | | | for DHA peak | Oxidation | The purity factor | Pass |
| | | | in final | Degradation | is within the | |
| | | | stressed test | | calculated | |
| | | | solutions. | | threshold limit | |
| 3. | Solution | Standard | For standard: | Standard Solution | | |
| | stability | solution and | Recovery for | Interval | Area | % Recovery |
| | | Test solution | solution | Initial | 437770 | 99.7 |
| | | | stability | 3^{rd} day | 5° C.-8° C. | 23° C.-27° C. |
| | | | standard | | 99.8 | 99.7 |
| | | | against freshly | | | |
| | | | prepared | | | |
| | | | standard | | | |
| | | | solution | | | |
| | | | should be in | | | |
| | | | the range of | | | |
| | | | 98.0% to | | | |
| | | | 102.0%. | | | |
| | | | For sample | | | Absolute |

| solution: | Interval | Area | % Assay | % Diff. | |
|---|---|---|---|---|---|
| Absolute | Initial | 7331860 | 100.6 | Not | |
| difference | | | | Applicable | |
| between % | 3^{rd} day | 5° C.-8° C. | | 23° C.-27° C. | |
| assay of n^{th} | | | Absolute | | Absolute |
| time point and | | % Assay | % Diff. | % Assay | % Diff. |
| initial time | | 100.9 | 0.3 | 101.2 | 0.6 |
| point should | | | | | |
| be NMT 2.0%. | | | | | |

| | | | | | | |
|---|---|---|---|---|---|---|
| 4. | LOD and | LOD & LOQ | Recorded the | Component | Concentration | Percentage |
| | LOQ | Prediction | results | Name | (μg/ml) | w/w |

| | | | |
|---|---|---|---|
| LOD | LOQ | LOD | LOQ |
| 0.742 | 2.250 | 1.484 | 4.500 |

| 5. | Linearity | Correlation | r should be | 0.99973 | | | |
|---|---|---|---|---|---|---|---|
| | | coefficient (r) | NLT 0.999 | | | | |
| | | Slope | Record the | 148112.06011 | | | |
| | | | results | | | | |
| | | Intercept | Record the | −24516.53392 | | | |
| | | | results | | | | |
| | | % deviation of | Should be not | −0.2 | | | |
| | | the Y- | more | | | | |
| | | intercept | than ±2.0% | | | | |
| 6. | Accuracy | Percentage | Each | | | | |
| | | recovery: | individual | % Level | % Recovery | Mean | % RSD |
| | | Level 50% | recovery and | 50% | 100.4 | 100.6 | 0.3 |
| | | | the mean | | 100.9 | | |
| | | | recovery at | | 100.5 | | |
| | | Level 100% | each level | 100% | 101.6 | 101.6 | 0.1 |
| | | | should be | | 101.7 | | |
| | | | between | | 101.6 | | |
| | | Level 200% | 98.0%- | 200% | 99.3 | 99.2 | 0.2 |
| | | | 102.0%. | | 99.0 | | |
| | | | Relative | | 99.4 | | |
| | | | standard | | | | |
| | | | deviation for | | | | |
| | | | % recovery of | | | | |
| | | | triplicate | | | | |
| | | | preparation | | | | |
| | | | (each level) | | | | |
| | | | should be not | | | | |
| | | | more than | | | | |
| | | | 2.0% | | | | |

| 7. | Range | Method is linear of 20% to 100%, precise and accurate in the range of 50% to 200% | | |
|---|---|---|---|---|
| | | of test concentration. | | |
| 8. | Precision | System | % RSD of DHA peak from six replicate | 0.1 |
| | | precision | injections of standard | |
| | | Standard | Solution-1 is NMT 2.0%. | |
| | | solution | | |
| | | Method | % Assay should comply as per | Complies as per |
| | | Precision | specification. | specification |
| | | | % Relative standard deviation (% RSD) | % RSD (Method |
| | | | of 6 samples should be NMT 2.0% | precision) |
| | | | | Assay |
| | | | | 0.6 |
| | | Inter mediate | % Assay should comply as per | Complies as per |
| | | Precision | specification. | specification |
| | | | % Relative standard deviation (% RSD) | % RSD (Intermediate |
| | | | of 6 samples should be NMT 2.0% | precision) |
| | | | | Assay |
| | | | | 0.8 |
| | | | Overall relative standard deviation (RSD) | % RSD (Method |
| | | | of 12 samples from method precision | precision and |
| | | | and intermediate precision should be not | intermediate precision) |
| | | | more than 2.0%. | 0.6 |

| | | | | |
|---|---|---|---|---|
| 9. | Robustness | Standard | System suitability should | System suitability parameters were |
| | | solution | pass. | met as per the set acceptance |
| | | | | criteria |

| Flow rate variation | Robustness parameters were met as |
|---|---|
| Wavelength variation | per the set acceptance criteria |
| Column temperature Variation | |

| | | | | |
|---|---|---|---|---|
| 10. | System | Standard | 1. % RSD of area ratio of DHA | System suitability |
| | suitability | solution | (Docosahexaenoic acid) peak from five | parameters were met |
| | | | replicate injections of standard solution-1 is | as per the set |
| | | | not more than 2.0. | acceptance criteria |
| | | | 2. Calculate the % similarity factor of | for all the validation |
| | | | standard-2 against standard-1 as per the | parameters. |
| | | | following formula. The % Similarity factor | |
| | | | should be between 98.0%-102.0%. | |
| | | | 3. Asymmetry (Tailing factor) for DHA | |
| | | | (Docosahexaenoic acid) peak is not more | |
| | | | than 2.0. | |
| | | | 4. Asymmetry (Tailing factor) in each | |
| | | | condition is not more than 2.0. | |

1. An oral Omega-3-fatty acid-rich oil emulsion composition for use in management of treatment of a disorder to overcome the same.

2. The oral Omega-3-fatty acid-rich oil emulsion composition according to claim 1, wherein the Omega-3-fatty acid-rich oil is microalge oil containing 40% DHA and the emulsion comprising DHA 50-100 mg/ml.

3. The oral Omega-3-fatty acid-rich oil emulsion composition according to claim 1, wherein all ingredients are natural and biocompatible ingredients, compatible for administration in Children including neonates.

4. The oral Omega-3-fatty acid-rich oil emulsion composition according to claim 1, wherein size of the dispersed phase is in nanometric range.

5. The oral Omega-3-fatty acid-rich oil emulsion composition according to claim 1, wherein the same has stability at room temperature (about 30° C.) and refrigerated one (2-8° C.).

6. The oral Omega-3-fatty acid-rich oil emulsion composition according to claim 1, wherein the emulsion comprises Vitamins and Minerals in therapeutically effective amounts.

7. The oral Omega-3 fatty acid-rich oil emulsion composition according to claim 1, wherein the disorder is selected from a group consisting of pre-term birth disorder in pregnant women, cognitive disorders in children and cardiovascular disorders.

8. The oral Omega-3 fatty acid-rich oil emulsion composition according to claim 7, wherein:
a. pre-term birth disorder in pregnant women is overcome by achieving normal delivery,
b. cognitive disorders in children is overcome by improvement in their cognitive ability, and
c. cardiovascular disorder is overcome by return to healthy condition.

9. An oral Omega-3 fatty-acid-rich oil thixotropic emulsion in nanomeric size range and having better absorption the oral Omega-3-fatty-acid-rich oil thixotropic emulsion according to claim 4 comprising emulsion in nanometric size range having higher surface area and absorption.

10. The oral Omega-3-fatty-acid-rich oil thixotropic emulsion according to claim 9 comprising natural emulsifier and their derivatives and bio surfactants alone or in combination with vitamins, minerals, Generally Regarded As safe (GRAS) natural ingredients.

11. The oral Omega-3-fatty-acid-rich oil thixotropic emulsion according to claim 10, wherein the natural emulsifier comprises, one or more selected form the group consisting of natural gums, clays and polymers.

12. The oral Omega-3-fatty-acid-rich oil thixotropic emulsion according to claim 11, wherein the composition comprises additives further comprising one or more selected from the group consisting of rheology modifiers, anti-oxidants, preservatives, stabilizers, sweetening and flavoring agents.

13. An oral Omega-3 fatty-acid-rich oil emulsion composition having dispersed phase having nanometric size comprising natural emulsifier and their derivative surfactants alone or in combination with vitamins, minerals, Generally Regarded As safe (GRAS) natural ingredients.

14. The oral Omega-3-fatty-acid-rich oil emulsion according to claim 13, wherein the natural emulsifier comprises natural gums.

15. The oral Omega-3-fatty-acid-rich oil according to claim 13, wherein the composition comprises additives further comprising one or more selected form the group consisting of rheology modifiers, anti-oxidants, preservatives, stabilizers, sweetening and flavoring agents.

16. A High Performance Liquid Chromatography (HPLC) method for assay of omega-3 fatty acids.

17. The High Performance Liquid Chromatography (HPLC) method for assay of omega-3 fatty acids according to claim 16 comprising steps of:
a. separately injecting blank, standard solutions-1, standard solutions-2 and sample solution into the chromatograph,
b. recording the chromatograms, and
c. measuring the peak responses for Docosahexaenoic acid (DHA).

18. The High Performance Liquid Chromatography (HPLC) method for assay of omega-3 fatty acids according to claim 17, wherein: (a) the blank injected is in a single replicate, (b) standard solutions-1 injected are in five replicates, (c) standard solutions-2 injected are in two replicates, and (d) sample solution injected is in a single replicate, (e) column used is Thermo Syncronis C18 (250×4.6 mm), 5 μm or Equivalent, (f) pump mode is isocratic, (g) flow rate is 1.0 ml/min, (h) detection is at UV, 210 nm, (i) injection volume is 20 μl, (i) column oven temperature is 45° C., (k) run time is 20 minutes.

19. The High Performance Liquid Chromatography (HPLC) method for assay of omega-3 fatty acids according to claim 18, wherein:
a. the solution-1 comprises DHA working standard,
b. the solution-2 comprises DHA test solution,
c. sample solution comprises known quantity of DHA-rich algal oil sonicated for a period of time with n-Heptane in around bottom flask, adding Methanolic Sodium hydroxide solution and refluxed for 10 minutes with a stirrer, cooled in ice bath without removing the round bottom flask, slowly and cautiously adding Boron Triflouride Methanol Complex Solution, refluxing further with magnetic with stirrer, cooled in ice bath without removing the round bottom flask, slowly with cautiously adding n-heptane and refluxing, cooling the mixture and removing the round bottom flask, adding saturated Sodium chloride solution, shaking well and transferring the contents to a centrifuge tube. Centrifuging with low speed, diluting upper Heptane layer with Isopropyl Alcohol and mixing the same. Further diluting this solution with Methanol and mixing the same.

20. The oral Omega-3-fatty acid-rich oil emulsion according to claim 9 wherein:
a. the natural emulsifiers comprise one or more selected from the group consisting of
i. Vitamin E TPGS (d-α-Tocopheryl polyethylene glycol 1000 succinate);
ii. Phospholipids comprise one or more selected from the group consisting of soya- and egg phosphatidylcholine, distearyl phosphatidylcholine, phosphatidylethanolamine and phosphatidylserine;
iii. gums comprise one or more selected from the group consisting of gum acacia, guar gum, xanthan gum, and gum tragacanth; and
iv. polymers comprise, one or more selected from the group consisting of pectin, gelatin and alginate,
b. emulsion stabilizers comprise one or more selected from the group consisting of Xanthan gum, guar gum, gum acacia, Bentonite, glycerol and mixture thereof,
c. The oral Omega-3-fatty acid-rich oil comprise of one or more selected from the group consisting of microalgae oil, fish oil or flaxseeds oil.
d. antioxidants comprise one or more selected from the group consisting of butylated hydroxytoluene, rosemary oil, sodium ascorbate, Vitablend (consisting of Vitamin E and ascorbyl palmitate), sodium metabisulphite, ascorbyl palmitate and Vitamin E,
e. vitamins comprise one or more selected from the group consisting of oil soluble vitamin A, vitamin D, vitamin E, vitamin K; water soluble Vitamin B1, Vitamin B2, Vitamin B3, Vitamin B5, Vitamin B6, Vitamin B12, folic acid and vitamin C. Minerals include zinc, copper, magnesium, potassium, calcium as calcium phosphate or calcium carbonate, iron and β-carotene etc.
f. buffers comprise one or more selected from the group consisting of sodium citrate sodium carbonate and phosphate buffer flavoring agent comprise one or more flavors selected from the group consisting of orange, strawberry, raspberry, mango, peach, vanilla, lime flavors,
g. sweetening agents comprise one or more selected from the group consisting Sorbitol, xylitol, mannitol, Sucralose, Stevia, Aspartame, Neotame, Acesulfame K and mixtures thereof,
h. preservative comprises rosemary extract, sodium benzoate, sodium azide, Methyl and propyl Paraben.

21. A process of making oral Omega-3-fatty acid-rich oil emulsion comprising steps of:
a. making an oil phase of omega-3-fatty acid-rich microalgae oil by mixing a natural emulsifier, a blend of vitamins comprising an anti-oxidant with DHA-rich microalgae oil in a manufacturing tank with stirrer at room temperature
b. making an aqueous phase in a tank with stirrer comprising steps of:
i. soaking a gum in purified water for a period of time required for dissolution,
ii. dissolving Vitamin E TPGS (d-α-Tocopheryl polyethylene glycol 1000 succinate) in another vessel under mechanical stirring, and other water soluble ingredients comprising a preservative and a high intensity sweetener were mixed with this solution,
iii. thereafter, both gum and Vitamin E and Ascorbyl Palmitate solutions were mixed under mechanical stirring for a period of time required to form uniform mixture,
iv. thereafter, oil phase was added to the aqueous phase under mechanical stirring maintaining both phases at room temperature,
v. flavoring agent was added, and
vi. stirring was continued for a further period of time.

22. The oral Omega-3-fatty acid-rich oil emulsion according to claim 9 wherein:
a. the natural emulsifiers comprise one or more selected from the group consisting of
i. Vitamin E TPGS (d-α-Tocopheryl polyethylene glycol 1000 succinate);
ii. phospholipidstone or more selected from the group consisting of soya- and egg phosphatidylcholine, distearyl phosphatidylcholine, phosphatidylethanolamine and phosphatidylserine;
iii. gums consisting of gum acacia, guar gum, xanthan gum, and gum tragacanth; and
iv. polymers consisting of pectin, gelatin and alginate,
b. emulsion stabilizers comprise one or more selected from the group consisting of Xanthan gum, guar gum, gum acacia, Bentonite, glycerol and mixture thereof,
c. antioxidants comprise one or more selected from the group consisting of butylated hydroxytoluene, rosemary oil, sodium ascorbate, Vitablend (consisting of Vitamin E and ascorbyl palmitate), sodium metabisulphite, ascorbyl palmitate and Vitamin E,
d. vitamins comprise one or more selected from the group consisting of oil soluble vitamin A, vitamin D, vitamin E, vitamin K; water soluble Vitamin B1, Vitamin B2, Vitamin B3, Vitamin B5, Vitamin B6, Vitamin B12, folic acid and vitamin C. Minerals include zinc, copper, magnesium, potassium, calcium as calcium phosphate or calcium carbonate, iron and β-carotene etc.
e. buffers comprise one or more selected from the group consisting of sodium citrate and sodium carbonate,
f. flavoring agent comprise one or more flavors selected from the group consisting of orange, strawberry, raspberry, mango, peach, vanilla, lime flavors,
g. sweetening agents comprise one or more selected from the group consisting Sorbitol, xylitol, mannitol, Sucralose, Stevia, Aspartame, Neotame, Acesul fame K and mixtures thereof,
h. preservative comprises one or more selected from the group consisting of rosemary extract, sodium benzoate, sodium azide, Methyl and propyl Paraben,

23. The process of making oral Omega-3-fatty acid-rich oil emulsion according to claim 17, wherein,
a. the natural emulsifier comprises Soya phosphatidyl choline,
b. the oil phase comprises a mix of:
i. omega-3-fatty acid-rich microalgae oil 12.5-25% w/v,
ii. Anti-oxidant mix (Vitablend™) 0.05 to 0.5%,
iii. butylated hydroxytoluene 0.1%,
iv. DHA-rich microalgae oil 12.5-25% w/v,
v. the Manufacturing tank is stainless steel Jacketed,
vi. stirring is done at 40-50° C. with stirring speed of 100-300 RPM,
vii. the gum is Xanthan gum at 0.3-1.5% w/v),
viii. soaking was done in purified water at 40-50° C. for 1-5 h period,
ix. Vitamin E TPGS (d-α-Tocopheryl polyethylene glycol 1000 succinate) is added at 1-5% w/v), mechanical stirring is done at 1000-1500 RPM at 1000-1500 RPM, preservative is sodium benzoate 0.02-0.1% w/v,
x. high intensity sweetener is Sucralose 0.1-0.5% w/v),
xi. uniform mixture is done by stirring and mixing of both gum and Vitamin E TPGS solutions is done under mechanical stirring at 1000-1500 RPM at 40-50° C. for 30-60 minutes.
xii. finally, adding the oil phase was to the aqueous phase under mechanical stirring is done at 1000-1500 rpm, maintaining both phases at 40-50° C.,
xiii. flavor being added is orange oil at
0.5% w/v to 1.0% w/v and stirring continued at 1-2 hrs.

24. The oral Omega-3-fatty acid-rich oil emulsion according to claim 21, wherein:
a. the natural emulsifiers comprise one or more selected from the group consisting of
i. Vitamin E TPGS (d-α-Tocopheryl polyethylene glycol 1QGG succinate);
ii. Phospholipids comprise one or more selected from the group consisting of soya- and egg phosphatidylcholine, distearyl phosphatidylcholine, phosphatidylethanolamine and phosphatidylserine;
iii. gums comprise one or more selected from the group consisting of gum acacia, guar gum, xanthan gum, and gum targacanth, and
iv. polymers comprise, one or more selected from the group consisting of pectin, gelatin and alginate,
b. emulsion stabilizers comprise one or more selected from the group consisting of Xanthan gum, guar gum, gum acacia, Bentonite, glycerol and mixture thereof,
c. The oral Omega-3-fatty acid-rich oil comprise of one or more selected from the group consisting of microalgae oil, fish oil or flaxseeds oil.
d. antioxidants comprise one or more selected from the group consisting of butylated hydroxytoluene, rosemary oil, sodium ascorbate, Vitablend (consisting of Vitamin E and ascorbyl palmitate), sodium metabisulphite, ascorbyl palmitate and Vitamin E,
e. vitamins comprise one or more selected from the group consisting of oil soluble vitamin A, vitamin D, vitamin E, vitamin K; water soluble Vitamin B1, Vitamin B2, Vitamin B3, Vitamin B5, Vitamin B6, Vitamin B12, folic acid and vitamin C. Minerals include zinc, copper, magnesium, potassium, calcium as calcium phosphate or calcium carbonate, iron and b-carotene etc.
f. buffers comprise one or more selected from the group consisting of sodium citrate sodium carbonate and phosphate buffer flavoring agent comprise one or more flavors selected from the group consisting of orange, strawberry, raspberry, mango, peach, vanilla, lime flavors,
g. sweetening agents comprise one or more selected from the group consisting Sorbitol, xylitol, mannitol, Sucralose, Stevia, Aspartame, Neotame, Acesulfame K and mixtures thereof
h. preservative comprises rosemary extract, sodium benzoate, sodium azide, Methyl and propyl Paraben.

25. The oral Omega-3-fatty acid-rich oil emulsion according to claim 13, wherein:
a. the natural emulsifiers comprise one or more selected from the group consisting of
i. Vitamin E TPGS (d-α-Tocopheryi polyethylene glycol 1000 succinate);
ii. phospholipidstone or more selected from the group consisting of soya- and egg phosphatidylcholine, distearyl phosphatidylcholine, phosphatidylethanolamine and phosphatidylserine;
iii. gums consisting of gum acacia, guar gum, xanthan gum, and gum targacanth, and
iv. polymers consisting of pectin, gelatin and alginate,
b. emulsion stabilizers comprise one or more selected from the group consisting of Xanthan gum, guar gum, gum acacia, Bentonite, glycerol and mixture thereof,
c. antioxidants comprise one or more selected from the group consisting of butylated hydroxytoluene, rosemary oil, sodium ascorbate, Vitablend (consisting of Vitamin E and ascorbyl palmitate), sodium metabisulphite, ascorbyl palmitate and Vitamin E,
d. vitamins comprise one or more selected from the group consisting of oil soluble vitamin A, vitamin D, vitamin E, vitamin K; water soluble Vitamin B1, Vitamin B2, Vitamin B3, Vitamin B5, Vitamin B6, Vitamin B12, folic acid and vitamin C. Minerals include zinc, copper, magnesium, potassium, calcium as calcium phosphate or calcium carbonate, iron and b-carotene etc.
e. buffers comprise one or more selected from the group consisting of sodium citrate and sodium carbonate,
f. flavoring agent comprise one or more flavors selected from the group consisting of orange, strawberry, raspberry, mango, peach, vanilla, lime flavors,
g. sweetening agents comprise one or more selected from the group consisting Sorbitol, xylitol, mannitol, Sucralose, Stevia, Aspartame, Neotame, Acesulfame K and mixtures thereof,
h. preservative comprises one or more selected from the group consisting of rosemary extract, sodium benzoate, sodium azide, Methyl and propyl Paraben,

26. The oral Omega-3-fatty acid-rich oil emulsion according to claim 9, wherein:
a. the natural emulsifiers comprise one or more selected from the group consisting of
i. Vitamin E TPGS (d-α-Tocopheryi polyethylene glycol 1000 succinate);
ii. phospholipidstone or more selected from the group consisting of soya- and egg phosphatidylcholine, distearyl phosphatidylcholine, phosphatidylethanolamine and phosphatidylserine;
iii. gums consisting of gum acacia, guar gum, xanthan gum, and gum targacanth, and
iv. polymers consisting of pectin, gelatin and alginate,
b. emulsion stabilizers comprise one or more selected from the group consisting of Xanthan gum, guar gum, gum acacia, Bentonite, glycerol and mixture thereof,
c. antioxidants comprise one or more selected from the group consisting of butylated hydroxytoluene, rosemary oil, sodium ascorbate, Vitablend (consisting of Vitamin E and ascorbyl palmitate), sodium metabisulphite, ascorbyl palmitate and Vitamin E,
d. vitamins comprise one or more selected from the group consisting of oil soluble vitamin A, vitamin D, vitamin E, vitamin K; water soluble Vitamin B1, Vitamin B2, Vitamin B3, Vitamin B5, Vitamin B6, Vitamin B12, folic acid and vitamin C. Minerals include zinc, copper, magnesium, potassium, calcium as calcium phosphate or calcium carbonate, iron and b-carotene etc.
e. buffers comprise one or more selected from the group consisting of sodium citrate and sodium carbonate,
f. flavoring agent comprise one or more flavors selected from the group consisting of orange, strawberry, raspberry, mango, peach, vanilla, lime flavors,
g. sweetening agents comprise one or more selected from the group consisting Sorbitol, xylitol, mannitol, Sucralose, Stevia, Aspartame, Neotame, Acesulfame K and mixtures thereof,
h. preservative comprises one or more selected from the group consisting of rosemary extract, sodium benzoate, sodium azide, Methyl and propyl Paraben,
